(19) United States
(12) Patent Application Publication
(10) Pub. No.: US 2015/0082871 A1
(43) Pub. Date: Mar. 26, 2015

(54) ONBOARD DIAGNOSTIC OF ENGINE OIL QUALITY BY USING A TRANSFER FUNCTION WITH A PLURALITY OF FLUID PROPERTY SENSORS

(71) Applicant: Cummins Inc., Columbus, IN (US)

(72) Inventor: Yuhui ZHA, Columbus, IN (US)

(73) Assignee: Cummins Inc., Columbus, IN (US)

(21) Appl. No.: 14/033,323

(22) Filed: Sep. 20, 2013

(51) Int. Cl. G01N 33/28

(52) U.S. Cl. CPC G01N 33/2888
USPC 73/53.05

(57) ABSTRACT

A system and method for measuring engine oil quality is disclosed. The system and method include: a plurality of sensors detecting oil quality data; and a processor in communication with the plurality of sensors to receive the oil quality data and adapted to execute a transfer function to output qualitative and quantitative measurements with respect to contamination of the oil based upon the oil quality data.

### FIELD OF THE INVENTION

The present invention relates generally to systems, devices and methods for analyzing fluids and more specifically to systems, devices and methods for analyzing engine oil.

### BACKGROUND

As is well-known, oil is used as a lubricant for mechanical (particularly metallic) machines. More specifically, oil is widely used as a lubricant for engines used in cars, trucks and other vehicles.

Unfortunately, engine oil can become contaminated with a variety of pollutants including fuel, biodiesel, water and soot. These pollutants can severely undermine the performance characteristics of the fluid and lead to catastrophic engine failure. This is particularly problematic for the owners and operators of fleets of vehicles.

Two approaches are generally used in the art to address this problem. One approach involves the periodic replacement of engine oil based on duration of use or mileage considerations. However, this approach lacks in accuracy. Consequently, the oil may be replaced well beyond the ideal replacement period leading to substandard performance and engine degradation. On the other hand, the premature replacement of oil can be costly and wasteful with respect to oil use per se.

In short, the conventional systems and methods prior art does not appear to teach how to distinguish multiple contamination types from each other or how to quantify the levels of such contaminants. Accordingly, a need remains for a more accurate and effective system and method for assessing engine oil quality. Specifically, a need remains for a system or method for identifying and distinguishing the nature and level of multiple contamination types in motor oil and other fluids.

### SUMMARY

The need in the art is addressed by the system and method for measuring engine oil quality of the present invention. A system for measuring engine oil quality comprises a plurality of sensors detecting oil quality data; and a processor in communication with the plurality of sensors to receive the oil quality data and adapted to execute a transfer function to output qualitative and quantitative measurements with respect to contamination of the oil based upon the oil quality data.

### BRIEF DESCRIPTION OF THE DRAWINGS

FIG. 1 is a block diagram of an illustrative embodiment of a system in accordance with the present invention.

FIG. 2 is a process flow map illustrating the method of the illustrative embodiment of the present invention.

FIG. 3 is a flow diagram showing an illustrative embodiment of the transfer function of the method of the present invention.

### DETAILED DESCRIPTION

The present invention relates generally to systems, devices and methods for analyzing fluids and more specifically to systems, devices and methods for analyzing engine oil.

The following description is presented to enable one of ordinary skill in the art to make and use the invention and is provided in the context of a patent application and its requirements. Various modifications to the preferred embodiments and the generic principles and features described herein will be readily apparent to those skilled in the art. Thus, the present invention is not intended to be limited to the embodiments shown, but is to be accorded the widest scope consistent with the principles and features described herein.

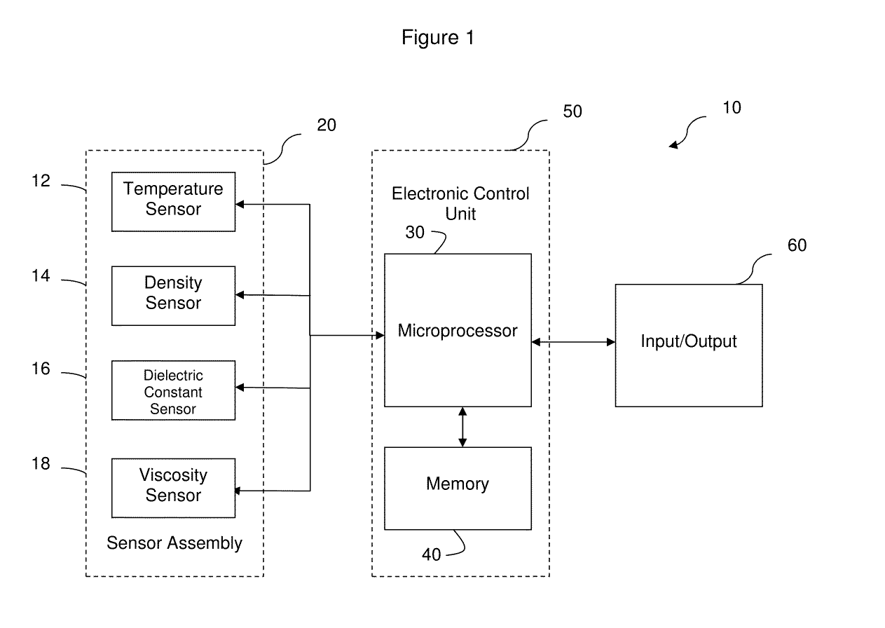

FIG. 1 is a block diagram of an illustrative embodiment of a system in accordance with the present invention. As shown in the Figure, the system 10 includes a temperature sensor 12, a density sensor 14, a dielectric constant sensor 16 and a viscosity sensor 18 mounted in a host vehicle or machine (not shown). In the illustrative embodiment, the sensors are implemented with a model FPS2800 sensor assembly 20 manufactured and sold by Measurement Specialty Inc. This assembly is adapted to measure temperature, density, viscosity and dielectric constant.

These data are provided to a microprocessor 30 adapted to execute a program or a lookup table stored in a tangible memory medium 40 to output qualitative measures with respect to at least four major oil contaminants including, in the illustrative embodiment, diesel fuel, biodiesel, water and soot.

Embodiments described herein can take the form of an entirely hardware implementation, an entirely software implementation, or an implementation containing both hardware and software elements. Embodiments may be implemented in software, which includes, but is not limited to, application software, firmware, resident software, microcode, etc.

The steps described herein may be implemented using any suitable controller or processor, and software application, which may be stored on any suitable storage location or computer-readable medium. The software application provides instructions that enable the processor to cause the receiver to perform the functions described herein.

Furthermore, embodiments may take the form of a computer program product accessible from a computer-usable or computer-readable medium providing program code for use by or in connection with a computer or any instruction execution system. For the purposes of this description, a computer-usable or computer-readable medium can be any apparatus that can contain, store, communicate, propagate, or transport the program for use by or in connection with the instruction execution system, apparatus, or device.

The medium may be an electronic, magnetic, optical, electromagnetic, infrared, semiconductor system (or apparatus or device), or a propagation medium. Examples of a computer-readable medium include a semiconductor or solid state memory, magnetic tape, a removable computer diskette, a random access memory (RAM), a read-only memory (ROM), a rigid magnetic disk, and an optical disk. Current examples of optical disks include DVD, compact disk-read-only memory (CD-ROM), and compact disk-read/write (CD-RAN).

The output from the microprocessor 30 can provide in high, medium and low classification levels that can provide a tiered alert on oil quality in terms of healthy, warning and condemning and also provide relative levels of these contaminants, which can point to specific engine problems and provide a guideline for maintenance. The microprocessor 30 may be included in an electronic control unit 50 or engine control module.

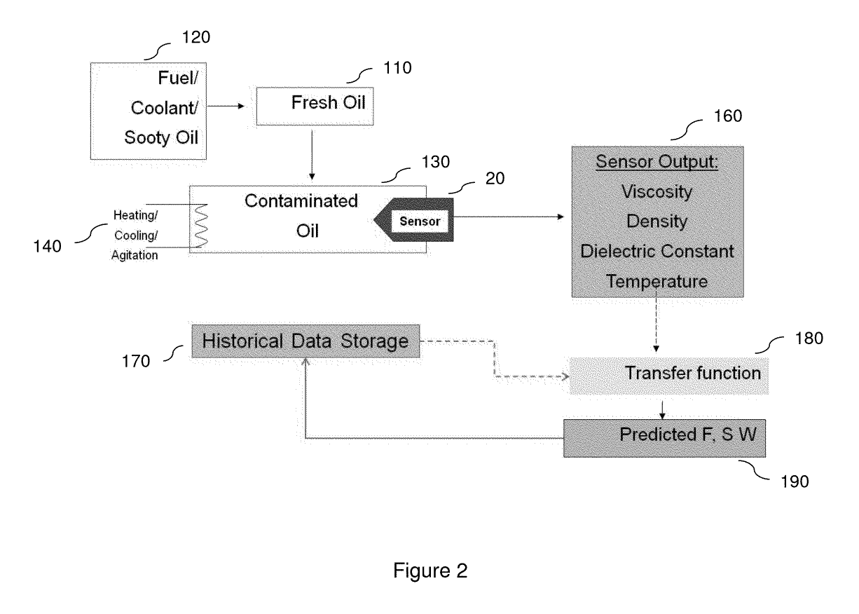

FIG. 2 is a process flow map illustrating the method of the illustrative embodiment of the system depicted in FIG. 1. As shown in FIG. 2, fresh oil 110 is contaminated with fuel, coolant, water and soot 120 resulting in contaminated oil 130. A host vehicle's engine provides heating, cooling and/or agitation as represented by the heating element 140. The sensor assembly 20 is coupled to the resulting heated, cooled, agitated contaminated oil and outputs a viscosity, density, dielectric constant and temperature data set 160. This data 160 are fed to a transfer function 180 along with the historical data 170 as initial value assigned to the parameters to be predicted, implemented via an algorithm discussed more fully below in software with coefficients stored in memory 40 and executed by the processor 30 in the illustrative embodiment of FIG. 1. As depicted in FIG. 2, the transfer function 180 provides predictions 190 of oil quality with respect to levels of fuel, soot and water contamination.

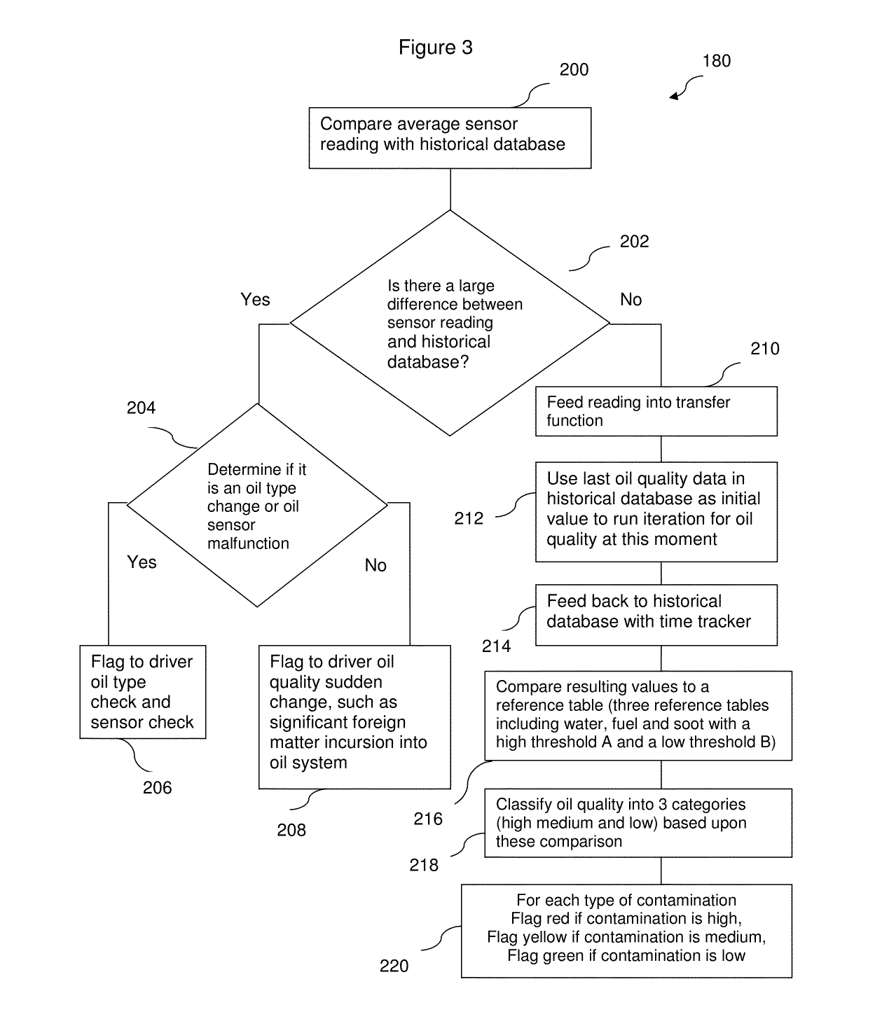

FIG. 3 is a flow diagram showing an illustrative embodiment of the transfer function 180 of the method of the present invention. An average sensor reading is compared with a historical database, via step 200. A determination is made on whether there is a large difference between the sensor reading and the historical database, via step 202.

If there is a large difference between the sensor reading and the historical database, a determination is then made on whether it is an oil type change or oil sensor malfunction, via step 204. If it is an oil type change or oil sensor malfunction, then oil type check and a sensor check are flagged to the driver, via step 206. If it is not an oil type change or oil sensor malfunction, then sudden oil quality change, such as significant foreign matter incursion into the oil system, is flagged to the driver, via step 208.

If there is not a large difference between the sensor reading and the historical database, the reading is fed into a transfer function, via step 210. Next, the last oil quality data in historical data base is used as initial value to run an iteration for oil quality at this moment, via step 212. The initial value is then fed back to historical database with a time tracker, via step 214.

In the illustrative embodiment, the coefficients shown in Table I below are used in equations [1-3] below to solve for and thereby predict the levels of fuel, soot and water contamination as three variables using Microsoft's Excel Solver addin, MATLAB or other equation solving utility known in the art:

TABLE I

| | V | D | DC |
|---|---|---|---|
| Predictor | Coef | Coef | Coef |
| Constant | 127.071 | 0.777159 | 2.23742 |
| F | −2.3058 | 0.002771 | 0.013012 |
| B | −11.9329 | 0.016678 | −0.16412 |
| S | 6.07014 | −0.00691 | 0.010789 |
| W | −17.2428 | 0.071881 | 1.0516 |
| T | −2.30702 | 0.001473 | −0.00333 |
| F*B | −3.69148 | 0.003506 | −0.01087 |
| F*S | −0.04001 | 0.000205 | 0.00854 |
| F*W | 0.41118 | −0.00059 | −0.01576 |
| B*S | −1.9884 | 0.002849 | −0.06978 |
| B*W | −49.046 | 0.040535 | 2.3904 |
| S*W | 0.3253 | −0.00486 | −0.03358 |
| F*B*S | 0.22608 | 1.44E−06 | 0.005393 |
| F*B*W | 15.2405 | −0.03727 | −0.21337 |
| B*S*W | 12.3516 | −0.01166 | −0.4639 |
| F*B*S*W | −3.25558 | 0.007752 | 0.020259 |
| F² | −0.01102 | −4.6E−05 | −0.00381 |
| B² | 16.4294 | −0.01969 | 0.1263 |
| S² | −0.05204 | −1.9E−05 | 0.024844 |
| W² | 7.0826 | −0.03783 | −1.14263 |
| T² | 0.011673 | −1.3E−05 | 1.86E−05 |
| F*T | 0.023582 | −2.8E−05 | 0.000174 |
| S*T | −0.05236 | 0.00014 | 0.00059 |
| B*T | 0.070726 | −0.00015 | −0.00082 |
| W*T | 0.115131 | −0.00035 | 0.007059 |
| F*S*T | 0.000252 | −2.7E−06 | −8.5E−05 |
| F*B*T | −0.00658 | 2.4E−05 | 9.39E−05 |
| F*W*T | −0.00429 | 1.26E−05 | 0.000424 |
| F*B*S*T | −0.00116 | −2E−06 | −2.6E−05 |
| F*B*W*T | 0.000559 | 0.000025 | 0.003595 |
| B*S*W*T | −0.06301 | −3.6E−05 | 0.005109 |
| B*S*T | 0.026375 | −5E−05 | −0.00067 |
| B*W*T | 0.13419 | 0.000362 | −0.03207 |
| S*W*T | −0.00456 | 7.31E−05 | −0.00135 |

where F is fuel, B is biodiesel, S is soot, W is water and T is temperature.

Hence,

Viscosity=127.071+(−2.3058)×F+(−11.9329)×B+6.07014×S+(−17.2428) ×W+(−2.30702)×T+(−3.69148)×F×B+(−0.04001)×F×S+0.41118×F×W+(−1.98884)×B×S+(−49.046)×B×W+(0.3253)×S×W+(0.22608)×F×B×S+(15.2405) ×F×B×W+(12.3516)×B×S×W+(−3.25558)×F×B×S×W+(−0.01102)×F\hat{0}2+(16.4294)×B\hat{0}2+(−0.05204)×S\hat{0}2+(7.0826)×W\hat{0}2+(0.011673)×T\hat{0}2+(0.023582)×F×T+(−0.05236)×S×T+(0.070726)×B×T+(0.115131)×W×T+(0.000252)×F×S×T+(−0.00658)×F×B×T+(−0.00429) ×F×W×T+(−0.00116)×F×B×S×T+(0.000559)×F×B×W×T+(−0.06301)XB×S×W×T+(0.026375)×B×S×T+(0.13419)×B×W×T+(−0.00456)×S×W×T [Equation 1]

Density=0.777159+(0.002771)×F+(0.016678)×B+(−0.00691)×S+(0.071881)×W+(0.001473)×T+(0.003506)×F×B+(0.000205)×F'S+(−0.00059)×F×W+(0.002849)×B×S+(0.040535)×B×W+(−0.00486)×S×W+(1.44E−06)×F×B×S+(−0.03727)×F×B×W+(−0.01166)×B×S×W+(0.007752)×F×B×S×W+(−4.6E−05)×F̂2+(−0.01969)×B̂2+(−1.9E−05) ×Ŝ2+(0.03783)×Ŵ2+(−1.3E−05) ×T̂2+(−2.8E−05)×F×T+(0.00014)×S×T++(−0.00015) ×B×T+(−0.00035)×W×T+(−2.7E−06)×F×S×T+(2.4E−05)×F×B×T+(1.26E−05)×F×W×T+(−2E−06)×F×B×S×T+(0.000025)×F×B×W×T+(−3.6E−05)XB×S×W×T+(−5E−05)×B×S×T+(0.000362)×B×W×T+(7.31E−05) ×S×W×T [Equation 2]

Dielectric constant=2.23742+(0.013012)×F+(−0.16412)×B+(0.010789)×S+(1.0516)×W+(−0.00333)×T+(−0.01087)×F×B+(0.00854)×F×S+(−0.01576F×W+(−0.06978)×B×S+(2.3904)×B×W+(−0.03358)×S×W+(0.005393) ×F×B×S+(−0.21337)×F×B×W+(−0.4639)×B×S×W+(0.020259) ×F×B×S×W+(−0.00381)×F̂2+(0.1263) ×B̂2+(0.024844)×Ŝ2+(−1.14263) ×Ŵ2+(1.86E−05)×T̂2+(0.000174)×F×T+(0.00059)×S×T++(−0.00082)×B×T+(.--7059)×W×T+(−8.5E−05)×F×S×T+(9.29E−05)×F×B×T+(0.000424)×F×W×T+(−2.6E−05)×F×B×S×T+(0.003595)×F×B×W×T+(0.005109)XB×B×S×W×T+(−0.00067)×B×S×T+(−0.03207)×B×W×T+(−0.00135)×S×W×T [Equation 3]

As stated above, the coefficients shown in Table I above are used in equations [1-3] above to solve for and thereby predict the levels of fuel, soot and water contamination in the oil. The resulting values of step 214 are then compared to a plurality of reference tables (in this embodiment there are three reference tables one for water contamination, one for fuel contamination and one for soot contamination, where each comparison has a high threshold value A and a low threshold value B), via step 216. Oil quality is classified into three categories (high, medium and low) based upon these comparisons, via step 218. Finally, for each type of contamination, a red indication is flagged if the contamination is high, a yellow indication is flagged if the contamination is medium, and a green indication is flagged if the contamination is low, via step 220.

Accordingly, a system and method in accordance with an embodiment provides a more accurate and effective system and method for assessing engine oil quality on a real time basis. Specifically, the invention provides a system or method for identifying and distinguishing the nature and level of multiple contamination types in motor oil and other fluids.

Although the present invention has been described in accordance with the embodiments shown, one of ordinary skill in the art will readily recognize that there could be variations to the embodiments and those variations would be within the spirit and scope of the present invention. Accordingly, many modifications may be made by one of ordinary skill in the art without departing from the spirit and scope of the present invention.

What is claimed is:

1. A system for measuring engine oil quality comprising:
a plurality of sensors for detecting oil quality data; and
a processor in communication with the plurality of sensors to receive the oil quality data and adapted to execute a transfer function to output qualitative and quantitative measurements with respect to contamination of the oil based upon the oil quality data.

2. The system of claim 1, wherein the plurality sensors include any combination of an oil temperature sensor; an oil density sensor; an oil dielectric constant sensor; and an oil viscosity sensor.

3. The system of claim 2, wherein said processor executes instructions to classify oil quality for each of the plurality of sensors into a plurality of categories based on the qualitative and quantitative measures.

4. The system of claim 2, wherein the plurality of categories include a high contamination category, a medium contamination category and a low contamination category.

5. The system of claim 1, wherein the qualitative and quantitative measures comprise any and any combination of fuel contamination; soot contamination; and water contamination.

6. The system of claim 1, wherein the qualitative and quantitative measures are compared to a plurality of reference tables; wherein the oil quality is determined based upon the comparison.

7. A computer implemented method for measuring engine oil quality comprising:
detecting oil quality data; and
utilizing a computer to execute a transfer function to output qualitative and quantitative measurements with respect to contamination of the oil based upon the oil quality data.

8. The computer implemented method of claim 7, wherein the detecting step comprises:
sensing oil temperature and providing temperature data in response thereto
sensing oil density and providing density data in response thereto;
sensing oil dielectric constant and providing dielectric constant data in response thereto; and
sensing oil viscosity and providing viscosity data in response thereto.

9. The system of claim 8, wherein the qualitative and quantitative measures comprise any and any combination of fuel contamination; soot contamination; and water contamination.

10. The system of claim 8, wherein the qualitative and quantitative measures are compared to a plurality of reference tables; wherein the oil quality is determined based upon the comparison.

11. A computer program product including a computer readable medium for measuring engine oil quality comprising, a computer readable program for causing a computer to control an execution of an application within a memory control device in operable communications with an engine; the computer readable program when executed by a computer for:
detecting oil quality data; and
executing a transfer function to output qualitative and quantitative measurements with respect to contamination of the oil based upon the oil quality data.

12. The computer program product of claim 11, wherein the detecting step comprises:
sensing oil temperature and providing temperature data in response thereto
sensing oil density and providing density data in response thereto;
sensing oil dielectric constant and providing dielectric constant data in response thereto; and
sensing oil viscosity and providing viscosity data in response thereto.

13. The system of claim 11, wherein the qualitative and quantitative measures comprise any and any combination of fuel contamination; soot contamination; and water contamination.

14. The system of claim 11, wherein the qualitative and quantitative measures are compared to a plurality of reference tables; wherein the oil quality is determined based upon the comparison.
